(19) United States
(12) Patent Application Publication
(10) Pub. No.: US 2026/0273234 A1
(43) Pub. Date: Sep. 17, 2026

(54) DUAL-LUMEN INTERMEDIATE SUPPORT CATHETER

(71) Applicants: Athos Patsalides, Greenvale, NY (US); Timothy White, Queens, NY (US); Daniel Toscano, Wantagh, NY (US)

(72) Inventors: Athos Patsalides, Greenvale, NY (US); Timothy White, Queens, NY (US); Daniel Toscano, Wantagh, NY (US)

(73) Assignee: IGNIS NEUROVASCULAR, INC., Greenvale, NY (US)

(21) Appl. No.: 19/077,250

(22) Filed: Mar. 12, 2025

(51) Int. Cl. A61M 25/09; A61M 25/00

(52) U.S. Cl. CPC A61M 25/09041; A61M 25/0026; A61M 2025/09008

(57) ABSTRACT

A dual-lumen intermediate support catheter with an intraluminal balloon system that includes two offset balloons adapted to inflate, contact, and hold an exchange wire placed through the catheter and therebetween. The balloons are formed from specific materials to form hydrophilic and hydrophobic portions for gripping the exchange wire in chosen ways for specific uses and purposes. Further, the balloons are respectively inflated with iodinated contrast to chosen pressures for specific uses and purposes as well. Furthermore, the exchange wire may be formed having sections of varying stiffness along its length for improved functionality, along with a surface on a proximal end portion thereof adapted to interact with the intraluminal balloons. Separate channels for balloon inflation may be formed within the wall or along the outer diameter of the intermediate support catheter. Once engaged, the exchange wire is used to navigate a micro-catheter into and out of position within a blood vessel of a patient.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71 (d).

### BACKGROUND OF THE INVENTION

### 1. Field of the Invention

This invention relates generally to neurointerventional support catheters, and more specifically to neurointerventional intermediate support catheters with intraluminal balloons.

### 2. Description of the Related Art

Catheterization is a cornerstone technique in all interventional specialties enabling minimally invasive access to the vascular system for diagnosis and treatment. During both diagnostic and treatment procedures, exchanging one catheter for another may be necessary to improve access or change devices. Catheter exchanges are pivotal in optimizing procedural outcomes, allowing clinicians to navigate complex vascular anatomy, deliver targeted therapies, and achieve efficient access to the desired treatment site. Catheter exchange techniques not only contribute to successful procedural outcomes but also plays a significant role in patient safety and complications.

In neurointerventional procedures, there are primarily two techniques for catheter and device exchange: over-the-wire exchange, and rapid or monorail exchange. Both techniques require the placement of a long stable microwire. With an over-the-wire exchange, a microcatheter is withdrawn over the wire while attempting to maintain distal wire position by holding the end of wire. In a monorail exchange, the distal portion of a catheter or device has a short secondary port which facilitates the exchange but decreases the stability and navigation of the device. Both techniques rely on a long exchange wire and open hemostatic valve exchanges, which pose significant procedural challenges and risks of vessel injury. The monorail technique is typically used in cardiology and has limited applications in brain vessels. In neurointerventional procedures, we use primarily over-the-wire exchanges.

The long exchange wire has significantly limited mobility, and inappropriate movement of the wire can result in vessel injury, vessel perforation, and loss of access. All of which can add significant risk to procedures. Vessel perforation, especially in the case of neurointerventional procedures, can be fatal. There is also a risk of losing vascular access during the exchange process. If the wire is inadvertently pulled out or displaced, it might require a more complicated procedure to regain access. Loss of access requires practitioners to re-cross or re-access lesions which may pose significant challenges or risk and can sometimes even be impossible to repeat. Furthermore, maintaining a wire in place while performing an exchange may lead to thrombus formation around the wire, particularly if there is prolonged access or compromised blood flow.

Currently, there are no neurointerventional intermediate catheters currently in use that can readily facilitate catheter exchange without the use of an exchange-length wire. We propose a novel intermediate catheter and wire system that would obviate the need for over-the-wire or monorail exchanges. This would then no longer limit practitioners' device choices and have the potential to significantly improve procedural safety.

Accordingly, the present invention overcomes the disadvantages associated with the prior art, by providing a dual-lumen intermediate support catheter with an intraluminal balloon system that includes two offset balloons adapted to inflate, contact, and hold an exchange wire placed through the catheter and therebetween.

### BRIEF SUMMARY OF THE INVENTION

In view of the foregoing disadvantages inherent in the known types of neurointerventional support catheters or the like in the prior art, the present invention provides a dual-lumen intermediate support catheter with an intraluminal balloon system that includes two offset balloons adapted to inflate, contact, and hold an exchange wire placed through the catheter and therebetween. The balloons are formed from specific materials to form hydrophilic and hydrophobic portions for gripping the exchange wire in chosen ways for specific uses and purposes. Further, the balloons are respectively inflated with iodinated contrast to chosen pressures for specific uses and purposes as well. Furthermore, the exchange wire may be formed having sections of varying stiffness along its length for improved functionality, along with a surface on a proximal end portion thereof adapted to interact with the intraluminal balloons. Separate channels for balloon inflation may be formed within the wall or along the outer diameter of the intermediate support catheter. Once engaged, the exchange wire is used to navigate a micro-catheter into and out of position within a blood vessel of a patient, with all the advantages of the prior art and none of the disadvantages.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

### BRIEF DESCRIPTION OF THE DRAWINGS

The figures which accompany the written portion of this specification illustrate embodiments according to the teachings of the present invention.

FIG. 1 shows a cut-away view of an exchange wire within the dual-lumen intermediate support catheter according to the preferred embodiment of the present invention.

FIG. 2 shows a cut-away view of the dual balloons inflated and contacting the exchange wire within the dual-lumen intermediate support catheter according to the preferred embodiment of the present invention of FIG. 1.

FIG. 3 shows a perspective view of the dual balloons inflated and contacting the exchange wire within the dual-lumen intermediate support catheter according to the preferred embodiment of the present invention of FIG. 1.

The various embodiments of the present invention will hereinafter be described in conjunction with the appended drawings.

### DETAILED DESCRIPTION

The embodiments of the present disclosure described below are not intended to be exhaustive or to limit the disclosure to the precise forms disclosed in the following detailed description. Rather, the embodiments are chosen and described so that others skilled in the art may appreciate and understand the principles and practices of the present disclosure.

The following embodiments and the accompanying drawings, which are incorporated into and form part of this disclosure, illustrate embodiments of the invention and together with the description, serve to explain the principles of the invention. To the accomplishment of the foregoing and related ends, certain illustrative aspects of the invention are described herein in connection with the following description and the annexed drawings. These aspects are indicative, however, of but a few of the various ways in which the principles of the invention can be employed and the subject invention is intended to include all such aspects and their equivalents. Other advantages and novel features of the invention will become apparent from the following detailed description of the invention when considered in conjunction with the drawings.

The instant inventive device includes two components. First, a dual-lumen intermediate support catheter with an intraluminal balloon system, including two offset balloons that are “surface-modified”. And second, an exchange wire formed having sections of varying stiffness along its length and a surface on a proximal end portion thereof adapted to interact with intraluminal balloons of the intraluminal balloon system.

The catheter would consist of either a laser-cut hypo-tube and/or a coil having a braid design, and include an inner and outer layer or coating. Between the inner and outer coatings two channels are formed. These channels would communicate with two intraluminal balloons. The balloons would be offset from one another at 180 degrees and spaced one immediately distal to the other between 5-25 cm proximal in the catheter. The distal portion of the catheter distal to the balloon would be flexible to support intracranial navigation, with the balloons being positioned more proximal to enhance stability. The balloons would each be marked with distal and proximal radiopaque markers to allow visualization during the procedure and facilitate the optimal position of the exchange wire.

The channels for the balloon inflation would be able to run along the outer diameter of the intermediate catheter or within the wall of the catheter and subsequently cross through the catheter. This would ensure that the balloon lumen would not interfere with the internal luminal diameter of the catheter. Each balloon would be surface-modified to enhance its ability to grip the catheter. Furthermore, inflation of the balloons would increase catheter stability. Each balloon would be inflated via a separate channel and side port and can be inflated separately or simultaneously. The balloons will have a predetermined volume so that the operators will know when the balloons are inflated according to the volume of fluid infused in the side port. The balloons will also be visible under fluoroscopy/X-ray when partially or fully inflated.

The exchange wire would be used to navigate a micro-catheter into position. Then, once the micro-catheter is withdrawn to the level of the balloons, a surface-modified portion of the exchange wire will be used to interact and grip the balloons. The exchange wire proximal to the balloon interaction segment would be surface-modified to enhance catheter sliding over the wire and navigation through the vasculature. The balloons would be inflated, and grip/lock the exchange wire in place. The micro-catheter would then continue to be removed without the use of an exchange wire. Once the next device is moved into position over the wire just proximal to the balloons, the balloons will be deflated to allow for passage of the device over the wire into position.

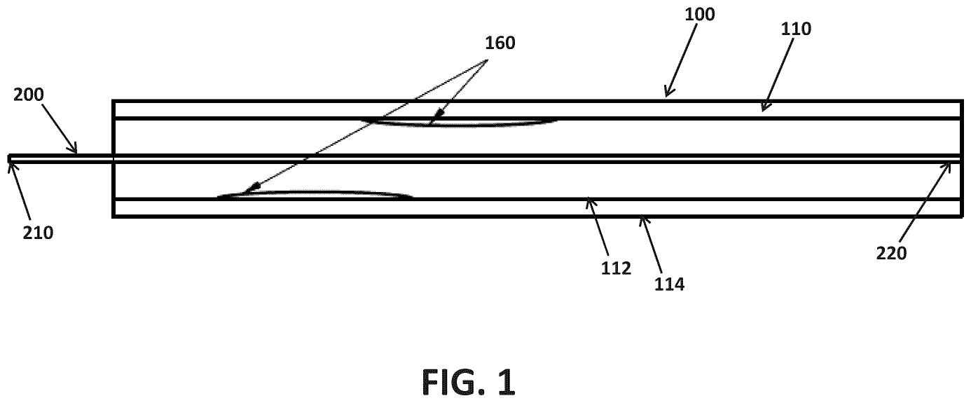

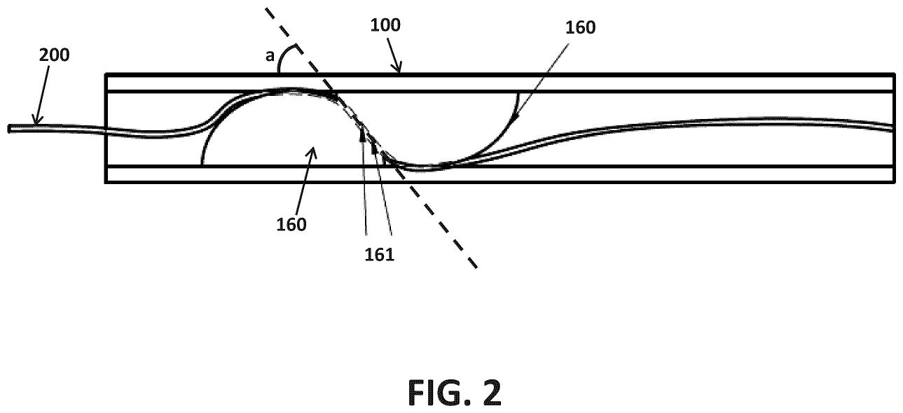

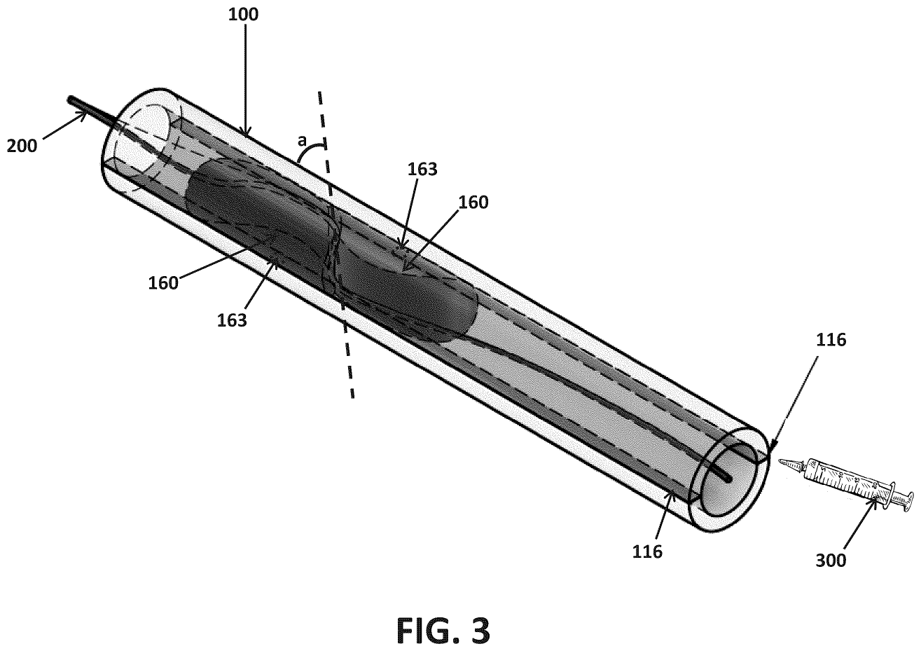

Turning now descriptively to FIGS. 1-3, the present invention discloses a combination of an exchange wire 200 and a dual-lumen intermediate support catheter 100. The exchange wire 200 includes an elongated body having a length, a distal end 210, and a proximal end 220, wherein the exchange wire 200 is adapted to navigate a micro-catheter into position within a catheter. The dual-lumen intermediate support catheter 100 comprises a catheter 110 including an elongated tubular body having an inner layer 112, and an outer layer 114, wherein the inner layer and the outer layer form two spaced elongated channels 116 extending along a length of the tubular body and adapted to allow fluid to pass respectively therethrough, and two balloons 160, wherein each of the two balloons 160 are connected to the inner layer 112 on opposite inner sides thereof and are adapted to receive the fluid from a respective channel 116 to thereby be inflated and deflated via an external fluid source 300, typically a fluid filled syringe inserted respectively into each channel, and wherein the two balloons 160 are connected to the inner layer 112 at differing distances along the length of the elongated tubular body, such that when the two balloons 160 are inflated they contact one another in an off-set manner, forming a surface angle (a) of between 45 and 60 degrees therebetween with respect to the length of the catheter, that is capable of contacting and releasably holding the exchange wire 200 in place within the elongated tubular body of the catheter, and such that when the two balloons 160 are deflated they release the exchange wire 200 such that the exchange wire can be moved and removed from the catheter.

The two balloons 160 are formed from a material 161 adapted to increase its ability to grip the exchange wire 200 when between the balloons and when the two balloons are inflated. The two channels 116 each include a side port 163 adapted to allow the fluid to pass therethrough and into and out of balloons 160. The distance between the two channels along the length of the elongated tubular body is between 5 and 25 centimeters. The two balloons 160 are each marked with distal and proximal radiopaque markers to allow visualization during procedures and facilitate an optimal position of an exchange wire. The balloon materials may include silicone, polyurethane elastomers (sometimes used for intracranial balloons for different applications), thermoplastic elastomer (TPE) (sometimes also used for intracranial balloons for different applications), nylon, and chronoprene all depending on the determined requirements. The balloons are also formed having radiopaque markers, one at the proximal end of a proximal balloon and one at the distal end of a distal balloons (2 markers), or each balloon's proximal and distal ends (4 markers). The balloons are inflated via the channels 116 extending to the end of the catheter (the part outside the body). The syringe 300 is then used to inflate (infuse contrast) and deflate (aspirate the contrast) the balloons. In most situations, iodinated contrast for balloon inflation will be used.

The elongated tubular body of the dual-lumen intermediate support catheter 100 may be formed from a braided material. Furthermore, the exchange wire 200 may further include surface portions adapted to interact and grip the two balloons 160. Furthermore, the exchange wire 200 may further include a portion formed from a material adapted to reduce friction between the exchange wire and the catheter, which includes nitinol, stainless steel, or a combination thereof. The surface portions of the wire may be formed to include hydrophobic properties which will increase friction between the wire and the balloons and increase stability during wire exchanges. Micro-textured surfaces and/or striations on the part of the wire that is in contact with the balloons will to increase friction and stability during exchanges as well. The wire is formed as being radiopaque, therefore there are radiolucent areas which are used to indicate which part of the wire should be aligned with the balloons.

Although specific embodiments have been illustrated and described herein, it will be appreciated by those of ordinary skill in the art that any arrangement, which is calculated to achieve the same purpose, may be substituted for the specific embodiment shown. This application is intended to cover any adaptations or variations of the present invention.

Although the invention has been explained in relation to its preferred embodiment, it is to be understood that many other possible modifications and variations can be made without departing from the spirit and scope of the invention.

What is claimed is:

1. A dual-lumen intermediate support catheter comprising:
a catheter including:
an elongated tubular body including:
an inner layer; and
an outer layer;
wherein said inner layer and said outer layer form two spaced elongated channels extending along a length of said tubular body and adapted to allow fluid to pass respectively therethrough; and
wherein each elongated channel includes a side port adapted to allow said fluid to pass in and out from said channels; and
two balloons;
wherein each of said two balloons are connected to said inner layer on opposite inner sides thereof, and are adapted to receive said fluid from a respective said channel to thereby be inflated and deflated via an external fluid source; and wherein said two balloons are connected to said inner layer at differing distances along the length of said elongated tubular body, such that when said two balloons are inflated they contact one another in an off-set manner forming a surface angle therebetween with respect to the length of said catheter and are capable of contacting and releasably holding an exchange wire placed within said elongated tubular body of said catheter, and such that when said two balloons are deflated they release said exchange wire such that said exchange wire can be moved and removed from said catheter.

2. The dual-lumen intermediate support catheter of claim 1, wherein said surface angle formed between said two balloons when inflated is between 45 and 60 degrees with respect to the length of said catheter.

3. The dual-lumen intermediate support catheter of claim 1, wherein said two balloons are formed from a material adapted to increase its ability to grip said exchange wire when between said balloons and when said two balloons are inflated.

4. The dual-lumen intermediate support catheter of claim 1, wherein said two channels each include a side port adapted to allow said fluid to enter therein.

5. The dual-lumen intermediate support catheter of claim 1, wherein the distance between said two channels along the length of said elongated tubular body is between 5 and 25 centimeters.

6. The dual-lumen intermediate support catheter of claim 1, wherein said two balloons are each marked with distal and proximal radiopaque markers to allow visualization during procedures and facilitate an optimal position of an exchange wire.

7. The dual-lumen intermediate support catheter of claim 1, wherein said elongated tubular body is formed from a braided material.

8. A combination of an exchange wire and a dual-lumen intermediate support catheter, said combination comprising:
an exchange wire including:
an elongated body having a length;
a distal end; and
a proximal end;
wherein said exchange wire is adapted to navigate a micro-catheter into position within a catheter;
a dual-lumen intermediate support catheter comprising:
a catheter including:
an elongated tubular body including:
an inner layer; and
an outer layer;
wherein said inner layer and said outer layer form two spaced elongated channels extending along a length of said tubular body and adapted to allow fluid to pass respectively therethrough; and
wherein each elongated channel includes a side port adapted to allow said fluid to pass in and out from said channels; and
two balloons;
wherein each of said two balloons are connected to said inner layer on opposite inner sides thereof, and are adapted to receive said fluid from a respective said channel to thereby be inflated and deflated via an external fluid source; and wherein said two balloons are connected to said inner layer at differing distances along the length of said elongated tubular body, such that when said two balloons are inflated they contact one another in an off-set manner forming a surface angle therebetween with respect to the length of said catheter that is capable of contacting and releasably holding said exchange wire in place within said elongated tubular body of said catheter, and such that when said two balloons are deflated they release said exchange wire such that said exchange wire can be moved and removed from said catheter.

9. The dual-lumen intermediate support catheter of claim 8, wherein said surface angle formed between said two balloons when inflated is between 45 and 60 degrees with respect to the length of said catheter.

10. The combination of claim 8, wherein said two balloons are formed from a material adapted to increase its ability to grip said exchange wire when between said balloons and when said two balloons are inflated.

11. The combination of claim 8, wherein said two channels each include a side port adapted to allow said fluid to enter therein.

12. The combination of claim 8, wherein the distance between said two channels along the length of said elongated tubular body is between 5 and 25 centimeters.

13. The combination of claim 8, wherein said two balloons are each marked with distal and proximal radiopaque markers to allow visualization during procedures and facilitate an optimal position of an exchange wire.

14. The combination of claim 8, wherein said elongated tubular body is formed from a braided material.

15. The combination of claim 8, wherein said exchange wire further includes a surface portion adapted to interact and grip said two balloons.

16. The combination of claim 15, wherein said exchange wire further includes a portion formed from a material adapted to reduce friction between said exchange wire and said catheter.
